(19) United States
(12) Patent Application Publication
(10) Pub. No.: US 2015/0328242 A1
(43) Pub. Date: Nov. 19, 2015

(54) IR-B Proprietary Blend

(71) Applicant: Wenhong Cao, Morrisville, NC (US)

(72) Inventor: Wenhong Cao, Morrisville, NC (US)

(21) Appl. No.: 14/276,418

(22) Filed: May 13, 2014

(51) Int. Cl. A61K 31/708; A61K 31/7076; A61K 31/341; A61K 31/198; A61K 31/353; A61K 31/7068; A61K 31/122

(52) U.S. Cl. CPC A61K 31/708; A61K 31/7068; A61K 31/7076; A61K 31/122; A61K 31/198; A61K 31/353; A61K 31/341

(57) ABSTRACT

The present invention provides methods of preventing insulin resistance including administering to a subject in need thereof an effective amount of a designated IR-B Proprietary Blend. IR-B Proprietary Blend can be used to antagonize/scavenge/reduce/diminish/blunt/inhibit/suppress reactive oxygen radicals (ROS). IR-B Proprietary Blend can be used to support/help/promote/enhance gene transcription. IR-B Proprietary Blend can be used to promote/improve/stimulate/enhance/amplify microcirculation. Insulin resistance or metabolic disorders that can be prevented by using the present invention include diabetes, hyperglycemia, insulin resistance, hyperinsulinemia, obesity, atherosclerosis, heart attack, stroke, hyperlipidemia and hyperlipoproteinemia, and accelerated aging.

### GOVERNMENT SUPPORT

This study was not supported by any government fund.

### FIELD OF THE INVENTION

The present invention relates to IR-B Blend that is a mixture of botanicals. IR-B represents “Insulin Resistance Beater” and can be used to antagonize reactive oxygen species (ROS) via multiple different mechanisms and consequently prevent insulin resistance, which is a precursor and/or key component of diabetes, obesity, metabolic syndrome, atherosclerotic heart and brain diseases (heart attack and stroke), Alzheimer's diseases, certain types of cancer, and aging.

### BACKGROUND OF THE INVENTION

USA CDC estimates that 25.6 million Americans (11.3%) have diabetes in 2010. The World Health Organization (WHO) estimates that more than 347 million People worldwide have diabetes (Lancet 378, 31-40). Diabetes can be characterized as a chronic condition that occurs when the pancreas does not produce enough insulin or when the body cannot effectively use the insulin it produces. The cause of diabetes is generally unknown, however, both genetics and environmental factors such as obesity and lack of exercise appear to be contributory factors.

In general, there are two basic forms of diabetes: Type I which results from the body's failure to produce insulin such that people with this type of diabetes produce very little or no insulin, and Type II which results from insulin resistance (a condition in which the body fails to properly use insulin), combined with relative insulin deficiency. A third type of diabetes, gestational diabetes mellitus (GDM), develops during some cases of pregnancy, but usually disappears after pregnancy. Pre-diabetes is a condition that occurs when a person's blood glucose levels are higher than normal but not high enough for a diagnosis of type II diabetes. It is estimated that 5-10% of Americans who are diagnosed with diabetes have type I diabetes, while most Americans who are diagnosed with diabetes have type II diabetes.

Typically, type II diabetes occurs approximately 13-15 years after insulin resistance has been established. Type I diabetes will also develop insulin resistance after treatment with insulin. Type III diabetes also have insulin resistance. Therefore, prevention of insulin resistance will not only prevent type II diabetes, but also improve the health in type I and type III diabetes.

Besides, insulin resistance may not only lead to type II diabetes, but may also cause atherosclerotic heart and brain diseases (heart attack and stroke), cancer, Alzheimer's diseases, and aging in the presence or absence of diabetes. Therefore, prevention of insulin resistance is important in prevention of diabetes and many other major health problems listed above.

In view of the foregoing, there is a need in the art for methods of preventing and reversing insulin resistance by all means.

### SUMMARY OF THE INVENTION

Embodiments of the present invention provide methods of preventing insulin resistance. Specifically, embodiments of the present invention relate to antagonizing oxidative stress, providing nutrients for gene transcription, and improving microcirculation.

According to some embodiments of the present invention, the mixture of botanicals can be used to prevent insulin resistance.

Embodiments of the present invention provide methods of antagonizing oxidative stress, i.e. reducing the level of reactive oxygen species (ROS).

Embodiments of the present invention further provide methods of providing necessary nutrients (nucleotides) for gene transcription.

Embodiments of the present invention also provide methods of improving microcirculation and then promoting structures and functions tissues and organs.

### Brief Description of IR-B Proprietary Blend

IR-B Blend is composed of the following botanicals: 1) mixed nucleotides (AMP, CMP, GMP, and UMP)(any amount except 0); 2) hematococcus pulvialis extracts containing astaxanthin (any amount except 0); 3) L-arginine (any amount except 0); 4) green tea extracts with epigallocatechin gallate (EGCG) and polyphenols (any amount except 0); and 5) Vitamin C (ascorbic acid)(any amount except 0). It can be in any form such as powder, liquid, semi-liquid, gel, gummy, solid tablets, and any other forms

### DETAILED DESCRIPTION

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods can be used for manipulation of nucleic acid sequences and the like according to the present invention. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, and other references mentioned herein are incorporated by reference in their entirety. However, the citation of a reference herein should not be construed as an acknowledgement that such reference is prior art to the present invention described herein.

As used herein, “a” or “an” or “the” can mean one or more than one. Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of 20%, 10%, 5%, 1%, 0.5%, or even 0.1% of the specified amount.

“Metabolic disorder” as used herein refers to diabetes, insulin resistance, glucose intolerance, hyperglycemia, hyperinsulinemia, obesity, hyperlipidemia, or hyperlipoproteinemia. The terms “diabetes” and “diabetes mellitus” are intended to encompass both insulin dependent and non-insulin dependent (Type I and Type II, respectively) diabetes mellitus, gestational diabetes, as well as pre-diabetes, unless one condition or the other is specifically indicated.

“Nucleic acid” or “nucleic acid sequence” as used herein encompasses both RNA and DNA, including cDNA, genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

By the term “express,” “expresses” or “expression” of a nucleic acid sequence, in particular a gluconeogenic nucleic acid sequence, it is meant that the sequence is transcribed, and optionally, translated. Typically, according to the present invention, transcription and translation of the coding sequence will result in production of a gluconeogenic polypeptide or active fragment thereof.

“Fragment” as used herein is one that substantially retains at least one biological activity normally associated with that protein or polypeptide. In particular embodiments, the “fragment” substantially retains all of the activities possessed by the unmodified protein. By “substantially retains” biological activity, it is meant that the protein retains at least about 50%, 60%, 75%, 85%, 90%, 95%, 97%, 98%, 99%, or more, of the biological activity of the native protein (and can even have a higher level of activity than the native protein).

“Variant” as used herein refers to nucleic acid molecules described herein that encode portions, analogs or derivatives of the gluconeogenic proteins described herein or the resulting protein product therefrom. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques, which include, but are not limited to oligonucleotide mediated mutagenesis, alanine scanning, PCR mutagenesis, site directed mutagenesis (see e.g., Carter et al., Nucl. Acids Res. 13:4331 (1986); and Zoller et al., Nucl. Acids Res. 10:6487 (1982)), cassette mutagenesis (see e.g., Wells et al., Gene 34:315 (1985)), restriction selection mutagenesis (see e.g., Wells, et al., Philos. Trans. R. Soc. London SerA 317:415 (1986)). Such variants include those produced by nucleotide substitutions, deletions or additions. The substitutions, deletions or additions may involve one or more nucleotides. The variants may be altered in coding regions, non-coding regions, or both.

“Subjects” as used herein are generally human subjects and include, but are not limited to, “patients.” The subjects may be male or female and may be of any race or ethnicity, including, but not limited to, Caucasian, African-American, African, Asian, Hispanic, Indian, etc. The subjects may be of any age, including newborn, neonate, infant, child, adolescent, adult, and geriatric. Subjects may also include animal subjects, particularly mammalian subjects such as canines, felines, bovines, caprines, equines, ovines, porcines, rodents (e.g. rats and mice), lagomorphs, primates (including non-human primates), etc., for treatment of metabolic disorders as well as veterinary medicine and/or pharmaceutical drug development purposes. Subjects further include, but are not limited to, those who are afflicted with or at risk for a metabolic disorder such as diabetes, insulin resistance, glucose intolerance, hyperglycemia, hyperinsulinemia, obesity, hyperlipidemia, or hyperlipoproteinemia. Risk factors for type II diabetes include, but are not limited to, obesity, apple-shaped figure, increased age, sedentary lifestyle, family history, history of diabetes in pregnancy, impaired glucose tolerance, ethnic ancestry, in particular, being of Aboriginal, African, Latin American or Asian ethnic ancestry increases the risk of developing of type II diabetes, high blood pressure and high cholesterol or other fats in the blood.

An “effective” amount as used herein is an amount of the compound or composition of this invention that provides some improvement or benefit to the subject. Alternatively stated, an “effective” amount is an amount that provides some alleviation, mitigation, or decrease in at least one clinical symptom of glucose intolerance or diabetes in the subject (e.g., improved glucose tolerance, enhanced glucose-stimulated insulin secretion, and the like) or in at least one clinical symptom of a disorder associated with hypersecretion of insulin or hyperproliferation of pancreatic islet beta cells (e.g., more normalized insulin levels, etc.) as is well-known in the art. Those skilled in the art will appreciate that the therapeutic effects need not be complete or curative, as long as some benefit is provided to the subject.

The term “regulate” as used herein refers to the ability to affect a method, process, state of being, disorder or the like. The effect may be that of prevention, treatment or modulation. “Modulate,” “modulates” or “modulation” refers to enhancement or inhibition in the specified activity. “Inhibition” or “inhibiting” refers to the prevention, reduction, decrease or cessation of the specified activity or process.

“Prevent,” “preventing” and “prevention” as used herein refer to an action resulting in a reduction or elimination in the chances of developing a certain condition described above. Thus, the term “prevent” refers to prophylactic regime.

According to embodiments of the present invention, methods of regulating a metabolic disorder include administering to a subject in need thereof an effective amount of IR-B Proprietary Blend. The metabolic disorder can be at least one of diabetes, hyperglycemia, insulin resistance, hyperinsulinemia, obesity, hyperlipidemia or hyperlipoproteinemia. In some embodiments, the metabolic disorder is diabetes. In some embodiments, the metabolic disorder is type I diabetes, type II diabetes, gestational diabetes or pre-diabetes. In other embodiments, the metabolic disorder is hyperglycemia.

The active agents can be formulated for administration in accordance with known pharmacy techniques. See, e.g., Remington, The Science And Practice of Pharmacy (9th Ed. 1995). In the manufacture of a nutritional compositions according to the present invention, the active agents (including the physiologically acceptable salts thereof) is typically admixed with, inter alia, an acceptable carrier. The carrier must, of course, be acceptable in the sense of being compatible with any other ingredients in the formulation and must not be deleterious to the subject. The carrier can be a solid or a liquid, or both, and can be formulated with the active agent as a unit-dose formulation, for example, a tablet, which can contain from 0.01% or 0.5% to 95% or 99%, or any value between 0.01% and 99%, by weight of the active agent. One or more active agents can be incorporated in the compositions of the invention, which can be prepared by any of the well-known techniques of pharmacy, comprising admixing the components, optionally including one or more accessory ingredients. Moreover, the carrier can be preservative free, as described herein above.

The nutritional compositions according to embodiments of the present invention include those suitable for oral, rectal, topical, inhalational (e.g., via an aerosol) buccal (e.g., sub-lingual), vaginal, topical (i.e., skin, ocular and mucosal surfaces, including airway surfaces), intraoperative, transdermal administration and parenteral (e.g., subcutaneous, intramuscular, intradermal, intraarticular, intrapleural, intraperitoneal, intraarterial, or intravenous), although the most suitable route in any given case will depend on the nature and severity of the condition being treated and on the nature of the particular active agent which is being used.

For example, formulations suitable for oral administration can be presented in discrete units, such as capsules, cachets, lozenges, or tablets, each containing a predetermined amount of the IR-B Proprietary Blend; as a powder or granules; as a solution or a suspension in an aqueous or non-aqueous liquid; or as an oil-in-water or water-in-oil emulsion. Such formulations can be prepared by any suitable method of pharmacy which includes bringing into association the active compound and a suitable carrier (which can contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet can be prepared by compressing or molding a powder or granules containing the active compound, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

As another example, formulations of the present invention suitable for parenteral administration comprise sterile aqueous and non-aqueous injection solutions of the active compound, which preparations are preferably isotonic with the blood of the intended recipient. These preparations can contain buffers and solutes that render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use. Extemporaneous injection solutions and suspensions can be prepared from sterile powders, granules and tablets of the kind previously described. For example, in one aspect of the present invention, there is provided an injectable, stable, sterile composition comprising active compounds, or a salt thereof, in a unit dosage form in a sealed container. The compound or salt is provided in the form of a lyophilizate that is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable can be employed in sufficient quantity to emulsify the compound or salt in an aqueous carrier. Non-limiting examples of agents that contribute to the pharmaceutical acceptability of the compositions of the present invention include normal saline, phosphatidyl choline, and glucose. In some embodiments, the pharmaceutically acceptable carrier can be normal saline. In other embodiments, the pharmaceutically acceptable carrier can be normal saline with up to 0.0, 0.01, 0.02, 0.03, 0.04, 0.05, 0.06, 0.07, 0.08, 0.09, 1, 2, 3, 4, 5, 6, 7, 8, 9, 10, 11, 12, 13, 14, 15, 16, 17, 18, 19, and 20%, and any value between 0.01% and 20%, glucose.

The effective amount of the IR-B Proprietary Blend will vary somewhat from subject to subject, and will depend upon factors such as the age and condition of the subject and the route of delivery. Such dosages can be determined in accordance with routine pharmacological procedures known to those skilled in the art. For example, the active agents of the present invention can be administered to the subject in an amount ranging from a lower limit of about 0.01, 0.02. 0.03, 0.04, 0.05, 0.06, 0.07, 0.08, 0.09, or 0.1 mg to an upper limit of about 0.1, 0.2, 0.3, 0.4, 0.5, 0.6, 0.7, 0.8, 0.9, 1, 2, 3, 4, 5, 6, 7, 8, 9, or 10 mg in a single dose; in an amount ranging from a lower limit of about 0.01, 0.02. 0.03, 0.04, 0.05, 0.06, 0.07, 0.08, 0.09, or 0.1 mg to an upper limit of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 mg in a 24 hour period. In some embodiments, the dosage can be in a range from about 0.1 mg/kg to about 100 mg/kg of total body weight of said individual. The frequency of administration can be one, two, three, four, five times or more per day or as necessary to control the condition. The duration of therapy depends on the type of condition being treated and can be for as long as the life of the subject.

The foregoing is illustrative of the present invention, and is not to be construed as limiting thereof. The invention is defined by the following claims, with equivalents of the claims to be included therein.

That which is claimed is:

1. The method of claim 1, wherein the mixture is a group of designated botanicals in IR-B Proprietary Blend.

2. The method of claim 1, wherein the mixture of the designated botanicals in IR-B Proprietary Blend is used to antagonized ROS (oxidative stress).

3. The method of claim 1, wherein the mixture of the designated botanicals in IR-B Proprietary Blend is used to improve microcirculation.

4. The method of claim 1, wherein the mixture of the designated botanicals in IR-B Proprietary Blend is used to provide necessary nutrients (mixed nucleotides) for gene transcription.

5. A method of claim 1, wherein the mixture of the designated botanicals in IR-B Proprietary Blend is used to prevent/treat insulin resistance (decreased response to insulin) and its many associated conditions such as metabolic syndrome/disorder, diabetes, hyperglycemia, hyperinsulinemia, obesity, hyperlipidemia or hyperlipoproteinemia.
